SEQUENCE LISTING (8 sequences)

SEQ ID NO: 1 - DNA, 75 bp, Artificial Sequence
  Artificial DNA Primer
caaagtatatgcctctccccagagtgttgcacctgtctccgtagcgtcacctcccggatgggagaaagtagactg

SEQ ID NO: 2 - DNA, 22 bp, Artificial Sequence
  Artificial DNA Primer
caaagtatatgcctctccccag

SEQ ID NO: 3 - DNA, 21 bp, Artificial Sequence
  Artificial DNA Primer
cagtctactttctcccatccg

SEQ ID NO: 4 - protein, 270 aa, Thermomyces lanuginosus
MEVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 5 - DNA, 75 bp, Artificial Sequence; identical to SEQ ID NO: 1
  Artificial DNA Primer

SEQ ID NO: 6 - DNA, 75 bp, Artificial Sequence
  Artificial DNA Primer
gctcccaaaagacctatcctgaccgcaaaatcataacgccccgacagcggtgtcttctccttcccagttcttacg

SEQ ID NO: 7 - DNA, 22 bp, Artificial Sequence
  Artificial DNA Primer
caaagtatatgcctctccccag

SEQ ID NO: 8 - DNA, 21 bp, Artificial Sequence
  Artificial DNA Primer
cagtctactttctcccatccg

(19) United States
(12) Patent Application Publication
(10) Pub. No.: US 2018/0073017 A1
(43) Pub. Date: Mar. 15, 2018

(54) METHODS FOR SELECTING ENZYMES HAVING ENHANCED ACTIVITY

(71) Applicant: NOVOZYMES A/S, Bagsvaerd (DK)

(72) Inventors: Robert Blazej, San Francisco, CA (US); Nicholas Toriello, San Carlos, CA (US); Charles Emrich, San Francisco, CA (US); Allan Svendsen, Hoersholm (DK)

(73) Assignee: NOVOZYMES A/S, Bagsvaerd (DK)

(21) Appl. No.: 15/563,707

(22) PCT Filed: Apr. 7, 2016
(86) PCT No.: PCT/US2016/026441

§ 371 Date: Oct. 2, 2017

Related U.S. Application Data
(60) Provisional application No. 62/143,967, filed on Apr. 7, 2015.

(51) Int. Cl. C12N 15/10; C12Q 1/68

(52) U.S. Cl. CPC C12N 15/1075; C12Q 1/6869; C12Q 1/6811; C12N 15/1058

(57) ABSTRACT

Provided herein are methods and means for enhancing enzymatic activity. The system makes use of an emulsion for in vitro compartmentalization of a library of synthetic compounds which have a gene and a marked enzymatic substrate both directly linked to a solid phase. Expressed enzymes with greater activity will preferentially release the selectable marker from the solid phase, whereas enzymes with less activity will leave the markers intact. Removal of the marked compounds provides an enriched gene library encoding for more active variants. Also described are synthetic compounds and emulsions which can be used in the methods.

### CROSS-REFERENCE TO RELATED APPLICATIONS

This application claims the priority benefit of U.S. Provisional Application No. 62/143,967, filed Apr. 7, 2015, the content of which is fully incorporated herein by reference.

### REFERENCE TO A SEQUENCE LISTING

This application contains a Sequence Listing in computer readable form, which is incorporated herein by reference.

### FIELD OF THE INVENTION

The present invention is in the technical field of protein engineering design and selection. More particularly, the present invention relates to enzyme enhancement by means of directed evolution.

### BACKGROUND

In vitro evolution using man-made compartments to link genotype and phenotype is a powerful system that has allowed the evolution of a vast range of molecules with diverse activities (See, e.g., Nat. Biotechnol., 1998, 16(7): 652-656). In vitro compartmentalization (IVC)-based selection was originally based on linking a DNA template directly to target substrate (WO1999/002671 A1). The gene-linked substrate is converted to product in the presence of expressed active enzyme, and the resulting product, which remains linked to the gene, is positively selected.

Blazej et al. disclosed another form of IVC-based self-selection in which DNA is linked directly to a solid-phase substrate which is capable of being degraded by an expressed active enzyme. Substrate degradation results in the active gene being self-released into solution, which is then isolated from inactive genes by negative selection (WO2009/124296 A2).

However, there remains a need for improved methods of selecting gene libraries for catalytic activity using substrates that resemble their native forms. The present invention fulfils that need.

### SUMMARY

Described herein are systems and components thereof for improving enzyme activity. Accordingly, in one aspect is a method of selecting for a polypeptide having enzyme activity, the method comprising:
(i) suspending a plurality of synthetic compounds in an aqueous phase, wherein the synthetic compounds individually comprise:
   (a) a polynucleotide encoding for a polypeptide;
   (b) a solid phase linked to said polynucleotide;
   (c) an enzyme substrate linked to said solid phase; and
   (d) a selectable marker linked to said substrate;
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion,
   wherein a polypeptide with enzymatic activity toward the substrate in an aqueous droplet releases the selectable marker(s) in that droplet; and
(iv) separating the synthetic compounds to recover synthetic compounds comprising the selectable marker and/or synthetic compounds wherein the selectable marker has been released.

In another aspect is a synthetic compound, comprising: (a) a polynucleotide encoding for a polypeptide; (b) a solid phase linked to said polynucleotide; (c) an enzyme substrate linked to said solid phase; and (d) a selectable marker linked to said substrate;

In another aspect is a method of making the synthetic compound, comprising: (i) linking the solid phase to the polynucleotide encoding for a polypeptide; (ii) linking the enzyme substrate to the solid phase; (iii) linking the selectable marker to the substrate; and (iv) recovering the synthetic compound.

In another aspect is a polynucleotide library, comprising a plurality of the synthetic compounds.

In another aspect is a water-in-oil emulsion, comprising the polynucleotide library, wherein the synthetic compounds of the library are compartmentalized in aqueous droplets of the emulsion.

In another aspect is a method of making the emulsion, comprising: (i) suspending the plurality of synthetic compounds in the aqueous phase, and (ii) mixing the suspension of (i) with an oil.

### BRIEF DESCRIPTION OF THE FIGURES

FIG. 1 shows an exemplary diagrammatic representation of the process steps involved in enzyme selection in accordance with one aspect of the present invention.

FIG. 2 shows an exemplary diagrammatic representation of a gold particle linked to a lipid enzyme substrate as described in Example 2.

FIG. 3 shows the differential capture of biotinylated-triglyceride-gold-nanoparticles with or without Thermomyces lanuginosus lipase (TLL), as described in Example 3. Gold nanoparticles were prepared with 0, 5, 10, and 20% LSS1b and dispersant. The nanoparticle were either digested with TLL (+TLL, black) or not digested (−TLL, white). Reactions were either performed in emulsions (emul.) or in free solutions (F/S).

FIG. 4 shows graph of the number of oligonucleotide molecules (DNA) per gold nanoparticle (AuNP) remaining on the nanoparticles after washing, as described in Example 4. Certain coupling conditions result in increased amounts of dithiol-modified oligonucleotides (SR1) compared to unmodified oligonucleotides (SR2), indicating specific conjugation of DNA to the nanoparticles.

### DEFINITIONS

Amino acid: The terms “amino acid” or “amino acid residue,” include naturally occurring L-amino acids or residues, unless otherwise specifically indicated. The terms “amino acid” and “amino acid residue” also include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Expression: The term “expression” includes the process of producing a polypeptide from a coding sequence, and may include but is not limited to, transcription, post-transcriptional modification, translation, post-translational modification, and secretion. Expression can be measured—for example, to detect increased expression—by techniques known in the art, such as measuring levels of mRNA and/or translated polypeptide. Expression, as used herein, includes in vitro transcription/translation.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, and the like with a nucleic acid construct or expression vector comprising a polynucleotide described herein (e.g., a polynucleotide encoding a polypeptide or polypeptide variant). The term “host cell” encompasses any progeny of a parent cell that is not identical to the parent cell due to mutations that occur during replication.

Linker: The term “linker” or “linked”, as used herein, refers to the chemical attachment of one referenced compound to another referenced compound.

Mutant: The term “mutant” means a polynucleotide encoding a variant.

Nucleic acid construct: The term “nucleic acid construct” means a nucleic acid molecule, either single- or double-stranded, which comprises one or more control sequences. The construct may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Parent or parent polypeptide: The term “parent” or “parent polypeptide” means a polypeptide to which an alteration is made to produce an enzyme variant. The parent may be a naturally occurring (wild-type) polypeptide or a variant or fragment thereof.

Polynucleotide: The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers to any form of DNA or RNA, including, for example, genomic DNA; complementary DNA (cDNA), which is a DNA representation of messenger RNA (mRNA), usually obtained by reverse transcription of mRNA or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands). Polynucleotides are said to be “different” if they differ in structure, e.g., nucleotide sequence.

Polypeptide: The term “polypeptide” refers to an amino acid polymer and is not meant to refer to a specific length of the encoded product and, therefore, encompasses peptides, oligopeptides, and proteins. The polypeptide may also be a naturally occurring allelic or engineered variant of a polypeptide.

Substrate: As used herein, the term “substrate” or “enzyme substrate” generally refers to a substrate for an enzyme; i.e., the material on which an enzyme acts to produce a reaction product. As used herein, the substrate is capable of releasing a linked selectable marker in the presence of an active enzyme.

Solid phase: As used herein, a “solid phase” refers to any material that is a solid when employed in the selection methods of the invention.

Synthetic compound: As used herein, the term “synthetic compound” refers to a compound that is not naturally occurring.

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position.

Wild-type: The term “wild-type” means an enzyme expressed by a naturally occurring microorganism, such as a bacterium, yeast, or filamentous fungus found in nature.

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

Described herein, inter alia, are methods and components used thereof for improving enzymatic activity. The invention employs in vitro compartmentalization (IVC) for rapid and high throughput enzyme evolution. Instead of relying on a physical link between the genotype and phenotype as implemented in display technologies, IVC links genotype and phenotype by spatial confinement in a single aqueous droplet of a water-in-oil emulsion (See, e.g., Tawfik, D. S. et al., Nat. Biotechnol., 1998, 16(7): 652-656; U.S. Pat. No. 6,489,103; WO 1999/002671; WO 2009/124296).

However, existing IVC screening systems require that the substrate must be directly linked with DNA. By modification with a large anionic macromolecule (˜660 kDa for a 1,000 bp DNA) the resulting substrate may be significantly altered and compromise selection relevancy.

As described herein, the Applicant has developed an improved IVC selection system wherein the substrate and gene are attached to a solid phase, and not directly to each other. The substrate also comprises a selectable marker (e.g. biotin, azide moiety, alkyne, protected maleimide, etc.) that can enable its identification and separation. Following gene expression, those polypeptides having enzymatic activity then cleave the marker from the substrate, allowing identification and separation of the cleaved (active) and uncleaved (less active or inactive) gene. Because the substrates are not directly attached to a polyanionic DNA, they better approximate native forms and are likely to result in the isolation of more commercially-relevant enzyme variants.

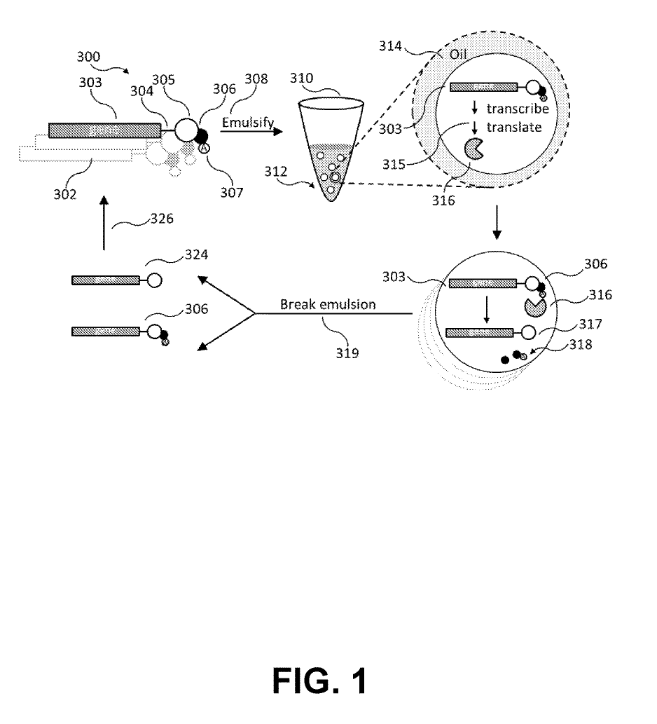

As exemplified by FIG. 1, a selection method (300) may employ a collection of polynucleotides (303) encoding for polypeptides, such as a library of synthetic compounds (302) comprising the polynucleotides. The polynucleotides of the library (303) are linked (304) to a solid phase (305) and are typically mutants that encode variants of an enzyme having activity toward the substrate (306), also linked to the solid phase (305). The polynucleotide mutants (303) of the library (302) encoding for the variants may be created using a variety of techniques including mutagenic PCR and DNA library synthesis as set forth in more detail below. PCR amplification using a modified PCR primer provides one means of linking (304) polynucleotide mutants (303) to solid phase (305). The substrate (306) is further linked to a selectable marker (307) which provides a means of selectively removing those synthetic compounds where the marker has not been released at the end of the process. The polynucleotide library (302) may be emulsified (308) using various oil-surfactants (314) with water to create an emulsion (310) containing aqueous droplets (312) (compartments), each with a compartmentalized synthetic compound. The emulsion is incubated to allow for expression (315) of the polynucleotide mutants (303) into corresponding polypeptides (316).

The expressed polypeptide variants (316) exhibiting activity toward the substrate (306) then release the selectable marker (307) from the substrate (318). Enzyme variants with enhanced activity are probabilistically more likely to release the substrate-bound marker (307) than variants exhibiting lower activity. A variable incubation temperature and time, as well as use of inhibitors and competitive substrates, enables tuning the assay stringency. After incubation, the emulsion (310) is broken (319). The synthetic compounds with substrate comprising a selectable marker are then separated from synthetic compounds where the selectable marker has been released (324) using techniques described herein (e.g., using affinity capture). Polynucleotide mutants that encode polypeptide variants with enhanced activity toward the substrate may be subjected to additional rounds (326) of selection to further enhance enzymatic activity.

Accordingly, in one aspect is a method of selecting for a polypeptide having enzyme activity (e.g., enhanced enzyme activity), the method comprising:
(i) suspending a plurality of synthetic compounds in an aqueous phase, wherein the synthetic compounds individually comprise:
   (a) a polynucleotide encoding for a polypeptide;
   (b) a solid phase linked to said polynucleotide;
   (c) an enzyme substrate linked to said solid phase; and
   (d) a selectable marker linked to said substrate;
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with enzymatic activity toward the substrate in an aqueous droplet releases the selectable marker(s) in that droplet; and (iv) separating the synthetic compounds to recover synthetic compounds comprising the selectable marker and/or synthetic compounds wherein the selectable marker has been released.

### Synthetic Compounds

In one aspect, the synthetic compounds used herein comprise (a) a polynucleotide encoding for a polypeptide, (b) a solid phase linked to the polynucleotide, (c) an enzyme substrate linked to the solid phase, and (d) a selectable marker linked to the substrate.

In some embodiments, a synthetic compound comprises one or more (e.g., two, three) copies of a polynucleotide (having the same or different sequence). In some embodiments, a synthetic compound comprises two polynucleotides (e.g., having the same or different sequence). In some embodiments, a synthetic compound comprises only one copy of one polynucleotide.

### Polynucleotides/Polypeptides

The polynucleotides may comprise a coding sequence for a polypeptide that is, or is derived from, a polypeptide having a desired enzymatic activity, such as, but not limited to, enzyme activity from the hydrolase class (EC 3) or lyase class (EC 4).

Non-limiting examples of polypeptides with enzymatic activity include aminopeptidases, amylases, amyloglucosidases, aspariginases, mannanases, carbohydrases, carboxypeptidases, cellulases, chitinases, cutinases, cyclodextrin glycosyltransferases, deoxyribonucleases, esterases, galactosidases, beta-galactosidases, glucoamylases, glucosidases, hemicellulases, invertases, lipases, lyases, mannosidases, pectinases, phytases, polyesterases, proteases, ribonucleases, and xylanases.

In one embodiment the polynucleotide encodes an amylase or a mannanase, in particular a variant of the amylase commercially available as Termamyl® or the mannanase commercially available as Mannaway® (both Novozymes AIS, Denmark). In another embodiment, the polynucleotide encodes a serine protease, for example, a subtilisin. In another embodiment, the polynucleotide encodes a maltogenic amylase. In another embodiment, the polynucleotide encodes a pullulanase.

The polynucleotides may comprise suitable control sequences, such as those required for efficient expression of the gene product, for example promoters, enhancers, translational initiation sequences, polyadenylation sequences, splice sites and the like, and as described in detail below.

As described supra, the methods of the present invention may comprise a plurality of synthetic compounds to create a polynucleotide library (e.g., a polynucleotide library encoding a library of enzyme variants). In particular embodiments, the libraries have at least about: 10², 10³, 10⁴, 10⁵, 10⁶, 10⁷, 10⁸, 10⁹, 10¹⁰, 10¹¹, 10¹², or 10¹⁴ different synthetic compounds and/or polynucleotides. Generally, the size of the library will be less than about 10¹⁵.

Libraries of polynucleotides can be created in any of a variety of different ways that are well known to those of skill in the art. In particular, pools of naturally occurring polynucleotides can be cloned from genomic DNA or cDNA (Sambrook et al., 1989); for example, phage antibody libraries, made by PCR amplification repertoires of antibody genes from immunized or unimmunized donors have proved very effective sources of functional antibody fragments (Winter et al., 1994; Hoogenboom, 1997). Libraries of genes can also be made by encoding all (see for example Smith, 1985; Parmley and Smith, 1988) or part of genes (see for example Lowman et al., 1991) or pools of genes (see for example Nissim et al., 1994) by a randomized or doped oligonucleotide synthesis.

Libraries can also be made by introducing mutations into a polynucleotide or pool of polynucleotides randomly by a variety of techniques in vivo, including; using mutator strains, of bacteria such as E. coli mutD5 (Liao et al., 1986; Yamagishi et al., 1990; Low et al., 1996); using the antibody hypermutation system of B-lymphocytes (Yelamos et al., 1995). Random mutations can also be introduced both in vivo and in vitro by chemical mutagens, and ionizing or UV irradiation (see Friedberg et al., 1995), or incorporation of mutagenic base analogues (Freese, 1959; Zaccolo et al., 1996). Random mutations can also be introduced into genes in vitro during polymerization for example by using error-prone polymerases (Leung et al., 1989). Further diversification can be introduced by using homologous recombination either in vivo (see Kowalczykowski et al., 1994) or in vitro (Stemmer, 1994a; Stemmer, 1994b)). Libraries of complete or partial genes can also be chemically synthesized from sequence databases or computationally predicted sequences.

Libraries can also be made using DNA recombination like e.g., DNA shuffling. Shuffling between two or more homologous input polynucleotides (starting-point polynucleotides) involves fragmenting the polynucleotides and recombining the fragments, to obtain output polynucleotides (i.e. polynucleotides that have been subjected to a shuffling cycle) wherein a number of nucleotide fragments are exchanged in comparison to the input polynucleotides. DNA recombination or shuffling may be a (partially) random process in which a library of chimeric genes is generated from two or more starting genes. A number of known formats can be used to carry out this shuffling or recombination process. The process may involve random fragmentation of parental DNA followed by reassembly by peR to new full-length genes, e.g. as presented in U.S. Pat. No. 5,605,793; U.S. Pat. No. 5,811,238; U.S. Pat. No. 5,830,721; U.S. Pat. No. 6,117,679. In-vitro recombination of genes may be carried out, e.g. as described in U.S. Pat. No. 6,159,687, WO98/41623, U.S. Pat. No. 6,159,688, U.S. Pat. No. 5,965,408, U.S. Pat. No. 6,153,510. The recombination process may take place in vivo in a living cell, e.g. as described in WO 97/07205 and WO 98/28416. The parental DNA may be fragmented by DNase I treatment or by restriction endonuclease digests as described by Kikuchi et al (2000a, Gene 236:159-167). Shuffling of two parents may be done by shuffling single stranded parental DNA of the two parents as described in Kikuchi et al (2000b, Gene 243:133-137). A particular method of shuffling is to follow the methods described in Crameri et al, 1998, Nature, 391: 288-291 and Ness et al. Nature Biotechnology 17: 893-896. Another format would be the methods described in U.S. Pat. No. 6,159,687: Examples 1 and 2.

### Solid Phases

The solid phase used with the synthetic compounds described herein may be any suitable solid phase known in the art. For example, materials useful as solid phases can include: natural polymeric carbohydrates and their synthetically modified, crosslinked, or substituted derivatives, such as agar, agarose, cross-linked alginic acid, chitin, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins, and keratins; natural hydrocarbon polymers, such as latex and rubber; synthetic polymers, such as vinyl polymers, including polyethylene, polypropylene, polystyrene, polyvinylchloride, polyvinyl acetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

Solid phases generally have a size and shape that permits their suspension in an aqueous medium, followed by formation of a water-in-oil emulsion. Suitable solid phases include microbeads or particles (both termed “microparticles” for ease of discussion). Microparticles useful in the invention can be selected by one skilled in the art from any suitable type of particulate material and include, but are not limited, to those composed of cellulose, Sepharose, polystyrene, polymethylacrylate, polypropylene, latex, polytetrafluoroethylene, polyacrylonitrile, polycarbonate, or similar materials. In some embodiments, the solid phase is a hydrophobic microbead (e.g., silica beads coated with C4, C8, and C18 alkyl groups, polystyrene, or PS-divinyl benzene).

Preferred microparticles include those averaging between about 0.01 and about 35 microns, more preferably between about 0.5 to 20 microns in diameter, haptenated microparticles, microparticles impregnated by one or preferably at least two fluorescent dyes (particularly those that can be identified after individual isolation in a flow cell and excitation by a laser), ferrofluids (i.e., magnetic particles less than about 0.1 micron in size), magnetic micro spheres (e.g., superparamagnetic particles about 3 microns in size), and other microparticles collectable or removable by sedimentation and/or filtration.

In some embodiments, the solid phase is a nanoparticle, such as a gold nanoparticle. Also contemplated are solid lipid nanoparticles, e.g., as described by Ekambaram et al. (Sci. Revs. Chem. Commun. 2012 2(1), 80-102. The nanoparticles are generally between about 1 to 400 nm in average diameter (e.g., 1 to 100 nm) and include, e.g., spherical colloidal gold, gold nanorods, and urchian shaped nanoparticles.

The solid phases are linked to the polynucleotides compounds by any means known to those in the art that do not interfere with expression. Examples of linking the solid phase to a polynucleotide can be found in WO 2009/124296 (the content of which is hereby incorporated by reference). Standard synthetic techniques may be employed, such as coupling the solid phase and polynucleotide using a reactive handle (e.g., an activated ester, azide, maleimide, etc.). In one example, the solid phase is coupled to a maleimide-linked oligonucleotide primer. The resulting conjugated oligonucleotide is then amplified by PCR with a template polynucleotide sequence to generate the desired synthetic compound. In another example, a 5′-thiol primer is coupled to a solid phase modified with a maleimide moiety, prior to PCR amplification to afford the desired synthetic compound. Similarly, an amino group on either a solid phase or polynucleotide can be linked to an activated ester (e.g., and NHS-ester) of the other partner to produce the desired synthetic compound. Even further still, the conjugation can employ click chemistry, for example, wherein an azide-modified solid phase is conjugated to an oligonucleotide primer having (i) a terminal alkyne for a copper(I) catalyzed [3+2] azide-alkyne cycloaddition (CuAAC), or (ii) a cyclooctyne derivative, such as dibenzocyclooctyl (DBCO), for a Cu-free click cycloaddition (Jewett et al. Chem. Soc. Rev. 2010 39(4):1272). Accordingly, in some embodiments, the polynucleotide encoding for a polypeptide is linked to the solid phase with a substituted thiol (e.g., thioether), substituted amino (e.g., amido), or triazole moiety.

### Substrates and Selectable Markers

The enzyme substrates used with the synthetic compounds described herein may be any suitable substrate as determined by the skilled artisan based on the desired enzymatic activity of the methods. For example, the substrate may be any substrate described supra for polypeptides with enzymatic activity, including but not limited to suitable substrates for aminopeptidases, amylases, amyloglucosidases, aspariginases, mannanases, carbohydrases, carboxypeptidases, cellulases, chitinases, cutinases, cyclodextrin glycosyltransferases, deoxyribonucleases, esterases, galactosidases, beta-galactosidases, glucoamylases, glucosidases, hemicellulases, invertases, lipases, lyases, mannosidases, pectinases, phytases, polyesterases, proteases, ribonucleases, and xylanases.

The selectable marker may be any suitable marker which is capable of being released from the substrate by an active enzyme to distinguish those compounds that have been altered by the active enzyme. As used herein a selectable marker is a chemical moiety that is capable of being released from the synthetic compound by a polypeptide having activity toward the substrate (e.g., by enzymatic hydrolysis), and which can be detected in a biochemical assay.

Suitable selectable markers include, but are not limited to affinity tags, where each affinity tag is a member of a binding pair. When used in the methods described herein, a synthetic compound comprising an affinity tag can further aid in separation from those compounds lacking the tag in step (iv), as compounds with the affinity tag are selectively removed by affinity capture.

Examples of binding pairs that may be used in the present invention include an antigen and an antibody or fragment thereof capable of binding the antigen, the biotin avidin/streptavidin pair (Savage et al., 1994), a calcium-dependent binding polypeptide and ligand thereof (e.g. calmodulin and a calmodulin-binding peptide (Stofko et al., 1992; Montigiani et al., 1996)), pairs of polypeptides which assemble to form a leucine zipper (Tripet et al., 1996), histidines (typically hexahistidine peptides) and chelated Cu²⁺, Zn²⁺ and Ni²⁺, (e.g. Ni-NTA; Hochuli et al., 1987), RNA-binding and DNA-binding proteins (Klug, 1995) including those containing zinc-finger motifs (Klug and Schwabe, 1995) and DNA methyltransferases (Anderson, 1993), and their nucleic acid binding sites. For example, suitable affinity tags include, inter alia, biotin, digoxigenin, dinitrophenyl (DNP), fluorescein, rhodamine (e.g., Texas Red®), and fucose. Biotin and fucose are capable of binding avidin and lectin, respectively, whereas digoxigenin, DNP, fluorescein, and rhodamine are capable of binding to product-specific antibodies. In one embodiment, the synthetic compound comprises a biotin selectable marker. In this embodiment, the one or more synthetic compounds comprising the selectable marker of step (iv) may be separated with streptavidin (e.g., streptavidin coated microspheres).

Conjugation of the enzyme substrate and selectable markers may be carried out using a variety of available conjugation techniques (e.g., those described supra and/or known in the art) and preferably does not interfere with activity of the enzyme on the substrate.

For example, to link the substrate to a solid phase, an amine modified enzyme substrate may be coupled to tosyl or carboxylate modified microspheres. Likewise, amino modified microspheres may be coupled to a tosyl or carboxylate modified enzyme substrate (or to an amino modified substrate compound via glutaraldehyde). Hydroxyl, hydrazide or chloromethyl modified microspheres can also be employed, as known in the art. Exemplary synthetic methods for linking the enzymes substrates to gold nanoparticles can be found in below (See, Example 2). In some embodiments, the substrate is linked to the solid phase with a substituted thiol (e.g., thioether), substituted amino (e.g., amido), or triazole moiety.

Additionally, to link the substrate to a selectable marker, a method related to amylases or amyloglucanases may use a starch substrate, such as maltoheptaose, conjugated to the reducing end of an amino-azide (e.g., or protected thiol, etc., in place of azide) via reductive amination. The activated linker is then modified by enzyme-catalyzed addition of a custom glucose 1-phosphate, X-amine (by phosphorylase) or UDP-GlcNac (by glycogen synthase). This will add a primary amine to the non-reducing end, which will then be used as a handle for attachment of a selectable marker, such as biotin. The reducing end can be coupled, e.g., via click chemistry (See supra) to a solid phase.

In another example, a method related to proteases may use a whole protein substrate which can be conjugated to pre-activated solid phase beads (e.g., epoxide or tosyl-activated magnetic polystyrene beads, such as Dynal M270 or M280), followed by reactivation of the protein with a reactive handle, such as an azide or thiol (e.g., using NHS-PEG₃-N₃ or SATA), to which the a selectable marker can be conjugated using methods described above.

In some embodiments, the selectable marker is linked to the substrate with a substituted thiol (e.g., thioether), substituted amino (e.g., amido), or triazole moiety.

Also contemplated are methods of making the synthetic compounds described herein, comprising: (i) linking the solid phase to the polynucleotide encoding for a polypeptide, (ii) linking the enzyme substrate to the solid phase, (iii) linking the selectable marker to the substrate, and (iv) recovering the synthetic compound.

### Formation of Aqueous Phases Containing Reagents for Polypeptide Expression

Synthetic compounds are combined in an aqueous phase with components for expression of the polypeptide (e.g., in vitro transcription/translation). Such components can be selected for the requirements of a specific system from the following: a suitable buffer, an in vitro transcription/replication system and/or an in vitro translation system containing all the necessary ingredients, enzymes and cofactors, RNA polymerase, nucleotides, transfer RNAs, ribosomes and amino acids (natural or synthetic).

A suitable buffer typically allows the desired components of the biological system to be active and will therefore depend upon the requirements of each specific reaction system. Buffers suitable for biological and/or chemical reactions are known in the art and recipes provided in various laboratory texts, such as Sambrook et al., 1989.

Exemplary in vitro translation systems can include a cell extract, typically from bacteria (Zubay, 1973; Zubay, 1980; Lesley et al., 1991; Lesley, 1995), rabbit reticulocytes (Pelham and Jackson, 1976), or wheat germ (Anderson et al., 1983). Many suitable systems are commercially available (for example from Promega) including some which will allow coupled transcription/translation (all the bacterial systems and the reticulocyte and wheat germ TNT™ extract systems from Promega). The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected (Ellman et al., 1991; Benner, 1994; Mendel et al., 1995).

### Formation of Emulsions

Emulsions may be produced from any suitable combination of immiscible liquids to enable a suitable platform for compartmentalizing the synthetic compounds described herein. In some embodiments, the emulsion is suitable for expressing the polypeptides (e.g., within an aqueous droplet), and those expressed polypeptides having enzymatic activity are capable of releasing the selectable marker from one or more synthetic compounds in that droplet.

Preferably the emulsion of the present invention has water (containing the biochemical components described supra) as the phase present in the form of finely divided droplets (the disperse, internal or discontinuous phase) and a hydrophobic, immiscible liquid (an oil) as the matrix in which these droplets are suspended (the nondisperse, continuous or external phase). Such emulsions are termed water-in-oil (W/O).

The emulsion may be stabilized by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash and Ash, 1993). Suitable oils include light white mineral oil and non-ionic surfactants (Schick, 1966) such as sorbitan monooleate (Span™ 80; ICI) and polyoxyethylenesorbitan monooleate (Tween™ 80; ICI).

The use of anionic surfactants may also be beneficial. Suitable surfactants include sodium cholate and sodium taurocholate. Particularly preferred is sodium deoxycholate, preferably at a concentration of 0.5% w/v, or below. Inclusion of such surfactants can in some cases increase the expression of the polynucleotides and/or the activity of the enzymes/enzyme variants. Addition of some anionic surfactants to a non-emulsified reaction mixture completely abolishes translation. During emulsification, however, the surfactant may be transferred from the aqueous phase into the interface and activity is restored. Addition of an anionic surfactant to the mixtures to be emulsified ensures that reactions proceed only after compartmentalization.

Creation of an emulsion generally requires the application of mechanical energy to force the phases together. There are a variety of ways of doing this that utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, ultrasound and ‘membrane emulsification’ devices (Becher, 1957; Dickinson, 1994). Accordingly, in one aspect is a method of preparing an emulsion described herein, comprising (i) suspending the plurality of synthetic compounds in the aqueous phase, and (ii) mixing the suspension of (i) with an oil.

Aqueous droplets formed in water-in-oil emulsions are generally stable with little if any exchange of polynucleotides or enzymes/enzyme variants between droplets. The technology exists to create emulsions with volumes all the way up to industrial scales of thousands of liters (Becher, 1957; Sherman, 1968; Lissant, 1974; Lissant, 1984).

The preferred droplet size will vary depending upon the precise requirements of any individual selection process that is to be performed according to the present invention. In all cases, there will be an optimal balance between polynucleotide library size, the required enrichment and the required concentration of components in the individual droplets to achieve efficient expression and reactivity of the enzymes/enzyme variants.

The processes of expression preferably occur within each individual droplet provided by the present invention. Both in vitro transcription and coupled transcription/translation become less efficient at sub-nanomolar DNA concentrations. Because of the requirement for only a limited number of DNA molecules to be present in each droplet, this therefore sets a practical upper limit on the possible droplet size. In some embodiments, the average volume of the droplets is between about 1 altoliter and about 1 nanoliter, inclusive (e.g., between about 10 altoliter and about 50 femtoliter, or about 0.5 femtoliter and about 10 femtoliter). The average diameter of the aqueous droplets typically falls within about 0.05 μm and about 100 μm, inclusive. In some embodiment, aqueous droplets in the emulsion have an average diameter between about 0.1 μm and about 50 μm, about 0.2 μm and about 25 μm, about 0.5 μm and about 10 μm, about 1 μm and about 5 μm, about 2 μm and about 4 μm, or about 3 μm and about 4 μm, inclusive. In certain embodiments, the mean volume of the droplets is less than 5.2×10⁻¹⁶ m³ (corresponding to a spherical droplet of diameter less than 10 μm), less than 6.5×10⁻¹⁷ m³ (corresponding to a spherical droplet of diameter less than 5 μm), less than or about 4.2×10⁻¹⁸ m³ (2 μm), or less than or about 9×10⁻¹⁸ m³ (2.6 μm).

The effective polynucleotide concentration in the droplets may be artificially increased by various methods that will be well-known to those versed in the art. These include, for example, the addition of volume excluding chemicals such as polyethylene glycols (PEG) and a variety of gene amplification techniques, including transcription using RNA polymerases including those from bacteria such as E. coli (Roberts, 1969; Blattner and Dahlberg, 1972; Roberts et al., 1975; Rosenberg et al., 1975), eukaryotes e.g. (Weil et al., 1979; Manley et al., 1983) and bacteriophage such as T7, T3 and SP6 (Melton et al., 1984); the polymerase chain reaction (peR) (Saiki et al., 1988); Q-beta replicase amplification (Miele et al., 1983; Cahill et al., 1991; Chetverin and Spirin, 1995; Katanaev et al., 1995); the ligase chain reaction (LCR) (Landegren et al., 1988; Barany, 1991); self-sustained sequence replication system (Fahy et al., 1991) and strand displacement amplification (Walker et al., 1992). Even gene amplification techniques requiring thermal cycling such as PCR and LCR could be used if the emulsions and the in vitro transcription or coupled transcription/translation systems are thermostable (for example, the coupled transcription/translation systems could be made from a thermostable organism such as Thermus aquaticus).

Increasing the effective local nucleic acid concentration enables larger droplets to be used effectively. This allows a preferred practical upper limit for most applications to the droplet volume of about 2.2×10⁻¹⁴ m³ (corresponding to a sphere of diameter 35 μm).

The droplet size should be sufficiently large to accommodate all of the required components of the biochemical reactions that are needed to occur within the droplet, in addition to the synthetic compound. In vitro, both transcription reactions and coupled transcription/translation reactions typically employ a total nucleotide concentration of about 2 mM. For example, in order to transcribe a gene to a single short RNA molecule of 500 bases in length, this would require a minimum of 500 molecules of nucleotides per droplet (8.33×10⁻²² moles). In order to constitute a 2 mM solution, this number of molecules must be contained within a droplet of volume 4.17×10⁻¹⁹ liters (4.17×10⁻²² m³ which if spherical would have a diameter of 93 nm.

Furthermore, the ribosomes necessary for the translation to occur are themselves approximately 20 nm in diameter. Hence, the in some embodiments lower limit for droplets is a diameter of approximately 0.1 μm (100 nm).

The size of emulsion droplets may be varied simply by tailoring the emulsion conditions used to form the emulsion according to requirements of the selection system. The larger the droplet size, the larger is the volume that will be required to emulsify a given polynucleotide library, since the ultimately limiting factor will be the size of the droplet and thus the number of droplets possible per unit volume. In some embodiments, the emulsion comprises at least about 10², 10³, 10⁴, 10⁵, 10⁶, 10⁷, 10⁸, 10⁹, 10¹⁰, 10¹¹, 10¹², or 10¹⁵ droplets/mL of emulsion.

Depending on the complexity and size of the library to be screened, it may be beneficial to form an emulsion such that in general 1 or less than 1 synthetic compound is included in each droplet of the emulsion. The number of synthetic compounds per droplet is governed by the Poisson distribution. Accordingly, if conditions are adjusted so that there are, on average, 0.1 synthetic compound per droplet, then, in practice, approximately: 90% of droplets will contain no synthetic compound, 9% of droplets will contain 1 synthetic compound, and 1% of droplets will contain 2 or more synthetic compounds. In practice, average values of about 0.1 to about 0.5, more preferably about 0.3, synthetic compounds per droplet provide emulsions that contain a sufficiently high percentage of droplets having 1 synthetic compound per droplet, with a sufficiently low percentage of droplets having 2 or more synthetic compounds per droplet. This approach will generally provide the greatest power of resolution. Where the library is larger and/or more complex, however, this may be less practical; it may be preferable to include several synthetic compound together and rely on repeated application of the method of the invention to achieve sorting of the desired activity. In some embodiments, no more than 70%, 60%, 50%, 40%, 30%, 20%, 15%, 10% or 5% of the aqueous droplets of the water-in-oil emulsion comprise more than one synthetic compound

Theoretical studies indicate that the larger the number of polynucleotide mutants created the more likely it is that a corresponding encoded polypeptide will be created with the properties desired (See, e.g., Perelson and Oster, 1979 for a description of how this applies to repertoires of antibodies). Recently it has also been confirmed practically that larger phage-antibody repertoires do indeed give rise to more antibodies with better binding affinities than smaller repertoires (Griffiths et al., 1994). To ensure that rare variants are generated and thus are capable of being selected, a large library size is generally desirable.

Using the present system, at an aqueous droplet diameter of 2.6 μm, a repertoire size of at least 10¹¹ can be readily sorted using 1 ml aqueous phase in a 20 ml emulsion.

### Expression, Separation and Further Processing

The emulsion is maintained for a sufficient time under conditions suitable for expression of the polypeptides. Expressed polypeptides having enzyme activity can release selectable marker (e.g., by enzymatic hydrolysis) from the synthetic compound (and corresponding gene) in that droplet. By attenuating the expression conditions using the teachings described herein, the gene coding sequences for those polypeptides with enhanced enzymatic activity can be distinguished from those having less activity.

In some embodiments, expression occurs by incubating the emulsion at about 25° C. to about 60° C. (e.g., about 25° C. to about 50° C., about 30° C. to about 40° C.) for about 1 hour to about 24 hours (e.g., about 1 hour to about 12 hours, about 1 hour to about 5 hours, or about 1 hour to about 2 hours).

In some embodiments, the aqueous phase is separated from the oil phase (e.g., prior to step (iv)) by any suitable technique, such as, for example chemically-induced coalescence and/or centrifugation.

The synthetic compounds comprising the selectable marker may be separated from those without the selectable marker using any of a number of conventional techniques. For example, as described supra, suitable antibodies, lectin, or streptavidin can bind to the marker and remove compounds containing the marker by affinity capture. In some embodiments, the recovered synthetic compounds comprising the selectable marker and/or those lacking the selectable marker results in the compounds being substantially pure. With respect to synthetic compounds wherein the selectable marker has been released (lacking the selectable marker), “substantially pure” intends a recovered preparation of synthetic compound that contains no more than 15% impurity, wherein impurity intends synthetic compound comprising the selectable marker. With respect to synthetic compounds comprising the selectable marker, “substantially pure” intends a recovered preparation of synthetic compound that contains no more than 15% impurity, wherein impurity intends synthetic compound lacking the selectable marker. In some variations, substantially pure synthetic compounds may contain no more than 10% impurity, or no more than 5% impurity, or no more than 3% impurity, or no more than 1% impurity, or no more than 0.5% impurity.

The collection of separated synthetic compounds (comprising and/or lacking the selectable marker) may be further analyzed. For example, after each round of selection, the enrichment of the pool of polynucleotides for those encoding a polypeptide of interest can be analyzed, e.g., by non-compartmentalized sequencing reactions known in the art. In one embodiment, the method further comprises analyzing the polynucleotide sequence (e.g., via sequencing) of one or more of the separated synthetic compounds of step (iv), such as one or more of the synthetic compounds comprising the selectable marker, and/or one or more of the compounds lacking the selectable marker.

The selected pool can be amplified and/or cloned into a suitable expression vector for propagation and/or expression, as described below, using techniques known in the art. In one embodiment, the method further comprises amplifying one or more polynucleotides of the synthetic compounds comprising the selectable marker in step (iv). In another embodiment, the method further comprises amplifying one or more polynucleotides of the synthetic compounds lacking the selectable marker in step (iv).

The polynucleotides of the separated synthetic compounds may also be subjected to subsequent, possibly more stringent rounds of sorting in iteratively repeated steps, reapplying the method of the invention either in its entirety or in selected steps only. By tailoring the conditions appropriately, synthetic compounds encoding enzymes having a better optimized activity may be generated after each round of selection. Accordingly, in some embodiments, the method is reiterated wherein the polynucleotides of the separated synthetic compounds (e.g., the amplified polynucleotides from the synthetic compounds) are used in a new plurality of synthetic compounds as described in step (i), and steps (i)-(iv) are repeated with said new plurality of synthetic compounds. If desired, further genetic variation can be introduced into the polynucleotides prior to repeating the method, using, e.g. error-prone polymerase chain reaction (PCR) and/or other techniques described supra. Accordingly, in one embodiment, the method further comprising introducing an alteration to (e.g., via mutagenizing) one or more polynucleotides of the separated synthetic compounds of step (iv).

### Nucleic Acid Constructs and Expression Vectors

In some embodiments, the methods described herein further comprise cloning one or more polynucleotides of the separated synthetic compounds from step (iv) into a nucleic acid construct or expression vector. RNA and/or recombinant protein can be produced from the individual clones for further purification and assay (as described below). Recombinant selected using the methods of the invention can be employed for any application for which the native enzyme is employed. Thus, in some embodiments, the methods further comprise expressing one or more of polynucleotides from the separated synthetic compounds of step (iv) (e.g., expressing a polynucleotide of a synthetic compound wherein the selectable marker has been released, thereby producing a polypeptide with enzymatic activity).

The nucleic acid constructs comprise a polynucleotide encoding a polypeptide or variant described herein operably linked to one or more control sequences that direct the expression of the coding sequence in a suitable host cell under conditions compatible with the control sequences.

The polynucleotide may be manipulated in a variety of ways to provide for expression of a polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol 3-hydroxypropionate dehydrogenase/glyceraldehyde-3-phosphate 3-hydroxypropionate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate 3-hydroxypropionate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol 3-hydroxypropionate dehydrogenase/glyceraldehyde-3-phosphate 3-hydroxypropionate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Thermomyces lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

Recombinant expression vectors comprise a polynucleotide encoding a polypeptide or variant described herein, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

In some embodiments, the methods described herein further comprise transforming one or more polynucleotides of the separated synthetic compounds from step (iv) (e.g., a nucleic acid construct or expression vector comprising the polynucleotide) into a recombinant host cell. A construct or vector comprising a polynucleotide is introduced into a host cell so that the construct or vector is maintained as a chromosomal integrant or as a self-replicating extra-chromosomal vector. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Thermomyces lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

In some embodiments, the methods described herein further comprise cultivating a recombinant host cell described supra under conditions suitable for expression of the polypeptide, and optionally recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

The present invention may be further described by the following numbered paragraphs:
[1] A method of selecting for a polypeptide having enzyme activity, the method comprising:
(i) suspending a plurality of synthetic compounds in an aqueous phase, wherein the synthetic compounds individually comprise:
   (a) a polynucleotide encoding for a polypeptide;
   (b) a solid phase linked to said polynucleotide;
   (c) an enzyme substrate linked to said solid phase; and
   (d) a selectable marker linked to said substrate;
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with enzymatic activity toward the substrate in an aqueous droplet releases the selectable marker(s) in that droplet; and
(iv) separating the synthetic compounds to recover synthetic compounds comprising the selectable marker and/or synthetic compounds wherein the selectable marker has been released.
   [2] The method of paragraph [1], wherein the plurality of synthetic compounds comprises at least about 10⁶ different synthetic compounds (e.g., at least about 10¹⁰, 10¹², or 10¹⁴ different synthetic compounds).
   [3] The method of paragraph [1] or [2], wherein no more than 20% of the aqueous droplets of the water-in-oil emulsion comprise more than one synthetic compound.
   [4] The method of any one of the preceding paragraphs, wherein each synthetic compound comprises only one copy of one polynucleotide.
   [5] The method of any one of the preceding paragraphs, wherein the emulsion comprises at least about 10⁶ aqueous droplets/mL of emulsion (e.g., at least about 10⁹, 10¹², or 10¹⁵ aqueous droplets/mL of emulsion).
   [6] The method of any one of the preceding paragraphs, wherein the aqueous droplets in the emulsion have an average diameter between about 0.05 μm and about 100 μm, inclusive (e.g., between about 0.1 μm and about 50 μm, about 0.2 μm and about 25 μm, about 0.5 μm and about 10 μm, or about 1 μm and about 5 μm, inclusive).
   [7] The method of any one of the preceding paragraphs, wherein the aqueous droplets in the emulsion have an average volume of between about 1 altoliter and about 1 nanoliter, inclusive (e.g., between about 10 altoliter and about 50 femtoliter, or about 0.5 femtoliter and about 10 femtoliter).
   [8] The method of any one of the preceding paragraphs, wherein the solid phase is linked to the polynucleotide with a substituted thiol (e.g., thioether), substituted amino (e.g., amido), or triazole moiety.
   [9] The method of any one of the preceding paragraphs, wherein the selectable marker is linked to the substrate with a substituted thiol (e.g., thioether), substituted amino (e.g., amido), or triazole moiety.
   [10] The method of any one of the preceding paragraphs, wherein the substrate is linked to the solid phase with a substituted thiol (e.g., thioether), substituted amino (e.g., amido), or triazole moiety.
   [11] The method of any one of the preceding paragraphs, wherein the selectable marker is an affinity tag.
   [12] The method of paragraph [11], wherein the affinity tag comprises biotin.
   [13] The method of paragraph [12], wherein the synthetic compounds comprising the selectable marker of step (iv) are separated with streptavidin (e.g., streptavidin coated microspheres).
   [14] The method of any one of the preceding paragraphs, wherein the solid phase is a microbead or particle.
   [15] The method of paragraph [14], wherein the solid phase is a hydrophobic microbead.
   [16] The method of any one of paragraphs [1]-[13], wherein the solid phase is a gold nanoparticle.
   [17] The method of any one of the preceding paragraphs, comprising separating the aqueous phase from the oil phase (e.g., via chemically-induced coalescence and/or centrifugation) prior to step (iv).
   [18] The method of any one of the preceding paragraphs, wherein the recovered synthetic compounds comprising the selectable marker and/or synthetic compounds wherein the selectable marker has been released are substantially pure.
   [19] The method of any one of the preceding paragraphs, further comprising analyzing the polynucleotide sequence (e.g., via sequencing) of the separated synthetic compounds of step (iv).
   [20] The method of any one of the preceding paragraphs, further comprising amplifying one or more polynucleotides of the synthetic compounds wherein the selectable marker has been released of step (iv).
   [21] The method of any one of the preceding paragraphs, further comprising amplifying one or more polynucleotides of the synthetic compounds comprising the selectable marker of step (iv).
   [22] The method of paragraph [20] or [21], wherein the amplified one or more polynucleotides are used in a new plurality of synthetic compounds as described in step (i), and steps (i)-(iv) are repeated with said new plurality of synthetic compounds.
   [23] The method of any one of the preceding paragraphs, further comprising introducing an alteration to (e.g., mutagenizing) one or more polynucleotides of the separated synthetic compounds of step (iv).
   [24] The method of paragraph [23], wherein the one or more altered polynucleotides are used in a new plurality of synthetic compounds as described in step (i), and steps (i)-(iv) are repeated with said new plurality of synthetic compounds.
   [25] The method of any one of the preceding paragraphs, further comprising expressing one or more of polynucleotides from the separated synthetic compounds of step (iv) (e.g., expressing a polynucleotide of a synthetic compound wherein the selectable marker has been released, thereby producing a polypeptide with enzymatic activity).
   [26] The method of any one of the preceding paragraphs, further comprising cloning one or more polynucleotides of the separated synthetic compounds from step (iv) into an expression vector.
   [27] The method of paragraph [26] further comprising transforming said expression vector into a recombinant host cell.
   [28] The method of paragraph [27], further comprising cultivating the recombinant host cell under conditions suitable for expression of the polypeptide, and optionally recovering the polypeptide.
   [29] A synthetic compound comprising:

(a) a polynucleotide encoding for a polypeptide;

(b) a solid phase linked to said polynucleotide;

(c) an enzyme substrate linked to said solid phase; and

(d) a selectable marker linked to said substrate.

[30] The synthetic compound of paragraph [29], which comprises only one copy of one polynucleotide.
[31] The synthetic compound of paragraph [29] or [30], wherein the solid phase is linked to the polynucleotide with a substituted thiol (e.g., thioether), substituted amino (e.g., amido), or triazole moiety.
[32] The synthetic compound of any one of paragraphs [29]-[31], wherein the selectable marker is linked to the substrate with a substituted thiol (e.g., thioether), substituted amino (e.g., amido), or triazole moiety.
[33] The synthetic compound of any one of paragraphs [29]-[32], wherein the selectable marker is linked to the substrate with a substituted thiol (e.g., thioether), substituted amino (e.g., amido), or triazole moiety.
[34] The synthetic compound of any one of paragraphs [29]-[33], wherein the selectable marker is an affinity tag.
[35] The synthetic compound of paragraph [34], wherein the affinity tag comprises biotin.
[36] The synthetic compound of any one of paragraphs [29]-[35], wherein the solid phase is a microbead or particle.
[37] The synthetic compound of paragraph [36], wherein the solid phase is a hydrophobic microbead.
[38] The synthetic compound of any one of paragraphs [29]-[35], wherein the solid phase is a gold nanoparticle.
[39] A method of making the synthetic compound of any one of paragraphs [29]-[38], comprising:

(i) linking the solid phase to the polynucleotide encoding for a polypeptide;

(ii) linking the enzyme substrate to the solid phase;

(iii) linking the selectable marker to the substrate; and

(iv) recovering the synthetic compound.

[40] A polynucleotide library comprising a plurality of different synthetic compounds according to any one of paragraphs [29]-[38].
[41] The polynucleotide library of paragraph [40], wherein the plurality of synthetic compounds comprises at least about 10⁶ different synthetic compounds (e.g., at least about 10¹⁰, 10¹², or 10¹⁴ different synthetic compounds).
[42] A water-in-oil emulsion comprising the polynucleotide library of paragraph [40] or [41], wherein the synthetic compounds are compartmentalized in aqueous droplets of the emulsion.
[43] The emulsion of paragraph [42], wherein no more than 20% of the aqueous droplets of the water-in-oil emulsion comprises more than one synthetic compound.
[44] The emulsion of paragraph [42] or [43], further comprising components for expression of the polypeptide in the aqueous droplets.
[45] The emulsion of paragraph any one of paragraphs [42]-[44], further comprising an emulsifying agent.
[46] The emulsion of any one of paragraphs [42]-[45], comprising at least about 10⁶ aqueous droplets/mL of emulsion (e.g., at least about 10⁹, 10¹², or 10¹⁵ aqueous droplets/mL of emulsion).
[47] The emulsion of any one of paragraphs [42]-[46], wherein the aqueous droplets have an average diameter between about 0.05 μm and about 100 μm, inclusive (e.g., between about 0.1 μm and about 50 μm, about 0.2 μm and about 25 μm, about 0.5 μm and about 10 μm, or about 1 μm and about 5 μm, inclusive).
[48] The emulsion of any one of paragraphs [42]-[47], wherein the aqueous droplets have an average volume of between about 1 altoliter and about 1 nanoliter, inclusive (e.g., between about 10 altoliter and about 50 femtoliter, or about 0.5 femtoliter and about 10 femtoliter).
[49] The emulsion of any one of paragraphs [42]-[48], wherein the emulsion is suitable for expressing the polypeptides within the aqueous droplets.
[50] The emulsion of any of one of paragraphs [42]-[49], wherein the expressed polypeptides having enzymatic activity toward the substrate are capable of releasing the selectable marker(s) from one or more synthetic compounds in that droplet.
[51] A method of making the emulsion of any one of paragraphs [42]-[50], comprising:

(i) suspending the plurality of synthetic compounds in the aqueous phase; and

(ii) mixing the suspension of (i) with an oil.

The following examples are provided by way of illustration and are not intended to be limiting of the invention.

### EXAMPLES

Chemicals used as buffers and substrates were commercial products of at least reagent grade.

### Example 1: Synthesis of LSS1b

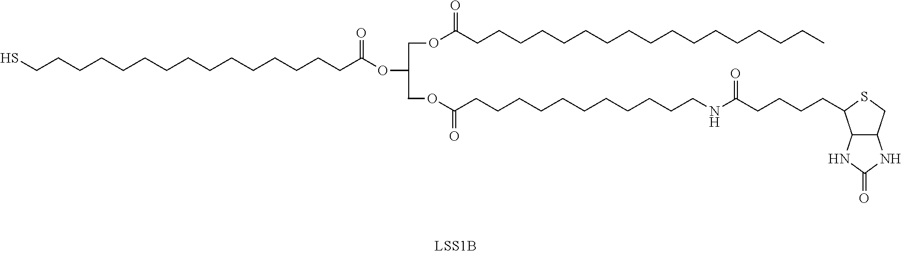

For the synthesis of LSS1b, 12-aminododecanoic acid (FA-NH2) was Mmt-protected similar to FA-SH (supra). Following a procedure for trityl protection of amino acids, in which the carboxylic acid is temporarily protected as a TMS-ester, the desired FA-NHMmt was produced in good yield (Barlos et al. J. Org. Chem. 1982, 47, 1324-1326). The coupling reactions now proceeded using the same protocol as for synthesis of LSS1. Since model studies indicated that biotin-NHS reacts fast and chemoselectively with amino nucleophiles, the DG-NHMmt was coupled with FA-SMmt, before both protecting groups were removed simultaneously. Finally, coupling to biotin-NHS yielded the desired LSS1b (2).

### Biotin-NHS

Biotin (325 mg, 1.33 mmol) was dissolved in DMF (10 mL) with gentle heating. After cooling to room temperature, N,N-diisopropylcarbodiimide (DIPCDI, 250 μL, 1 eq), pyridine (105 μL, 1 eq) and N-hydroxysuccinimide (HNS, 200 mg, 1.3 eq) were added. The mixture was stirred at room temperature overnight, then filtered and concentrated in vacuum. The resulting solid was re-crystallized in iPrOH (50 mL), by first dissolving it using gentle heating, then placing the solution to precipitate at 4° C. The precipitated product was filtered, washed with cold iPrOH and dried in vacuum to yield 280 mg (62%) of white crystals.

¹H NMR (400 MHz, DMSO-d6, selected signals in ppm): 6.42 (s, 1H, NH), 6.36 (s, 1H, NH), 4.33-4.29 (m, 1H, —CHNH—), 4.18-4.13 (m, 1H, —CHNH—), 3.15-3.08 (m, 1H, —CH—S—), 2.82 (s, 4H, —CH₂—NHS), 2.68 (t, 2H, —CH₂COO—).

¹³C NMR (100 MHz, DMSO-d6, selected signals in ppm): 170.7 (CO NHS), 169.4 (CO ester), 163.2 (CO carbamide), 61.5 (—CHNH—), 59.6 (—CHNH—), 55.7 (—CH—S—), 30.5 (—CH₂COO—), 25.9 (—CH₂—NHS).

### FA-NHMmt

12-Aminododecanoic acid (1.08 g, 5 mmol) was suspended in CHCl₃-MeCN (5:1, 18 mL) and chloro trimethylsilane (TMS-Cl, 0.63 mL, 1 eq) was added. The mixture was heated to reflux (65° C.) under N₂ for 2 h and remains in suspension. After cooling to room temperature, triethylamine (1.39 mL, 2 eq) was added. Then addition of Mmt-Cl (1.54 g, 1 eq) dissolved in CHCl₃ (10 mL). The turbid orange solution was stirred at room temperature overnight. MeOH (25 mmol, 1.0 mL) was then added. The orange solution slowly turned yellow. TLC (EtOAc-heptane, 1:3) confirmed full conversion of Mmt-Cl (Rf 0.40) to product (Rf 0.15). Evaporated to an oil, of which 1.7 g is purified by flash chromatography. It is dissolved in CHCl₃ (10 mL) and eluted through a 120 g silica flash cartridge using EtOAc-heptane (1:3, 600 mL and then 2:3, 750 mL). The product containing fractions were identified by TLC, pooled and evaporated to yield 1.4 g, 57% of yellow viscous oil.

¹H NMR (400 MHz, CDCl₃, selected signals in ppm): 6.83 (d, 2H, Mmt CH next to —OMe), 3.81 (s, 3H, —OMe), 2.36 (t, 2H, —CH₂COOH), 2.16 (t, 2H, —NHCH₂—), 1.68-1.61 (m, 2H, —CH₂CH₂COOH), 1.53-1.46 (m, 2H, —NHCH₂CH₂—).

¹³C NMR (100 MHz, CDCl₃, selected signals in ppm): 55.2 (—OMe), 43.7 (—NHCH₂—), 34.1 (—CH₂COOH).

### DG-NHMmt

FA-NHMmt (585 mg, 1.2 mmol) was dissolved in THF-DCM (1:1, 12 mL) and the solution cooled to 0° C. on ice. 1-Stearoyl-rac-glycerol (430 mg, 1 eq), EDAC (570 mg, 2.5 eq) and DMAP (26 mg, 0.2 eq) were added. The mixture was stirred under N₂ at 0° C. for 2 h, and then at room temperature for 2 h. Some precipitation. TLC (EtOAc-hexane 1:3) showed full conversion. Rf (product)=0.47. The reaction mixture was evaporated and re-dissolved in CHCl₃ (2 mL). Purification by flash chromatography, eluting with a hexane-EtOAc gradient going from pure hexane to 20% EtOAc. The product is eluted with 20% EtOAc. The product containing fractions are evaporated to yield 405 mg, 41%.

¹H NMR (400 MHz, CDCl₃, selected signals in ppm): 6.83 (d, 2H, Mmt CH next to —OMe), 4.23-4.09 (m, 5H, glycerin backbone), 3.80 (s, 3H, —OMe), 2.37 (t, 4H, —CH₂CO—), 2.13 (t, 2H, —CH₂NH—), 1.67-1.61 (m, 4H, —CH₂CH₂COOH), 1.55-1.45 (m, 2H, —NHCH₂CH₂—), 0.91 (t, 3H, —CH₃).

¹³C NMR (100 MHz, CDCl₃, selected signals in ppm): 173.9 (—CO—), 113.0 (Mmt CH next to —OMe), 70.3 (Mmt quaternary C), 68.4 (C-2), 65.06 (C-1/3), 55.2 (—OMe), 43.6 (—CH₂NH—), 34.1 (—CH₂CO—), 24.9 (—CH₂CH₂CO—), 22.7 (—CH₂CH₃), 14.1 (—CH₃).

### Example 2: Linking Enzyme Substrate to Solid Phase

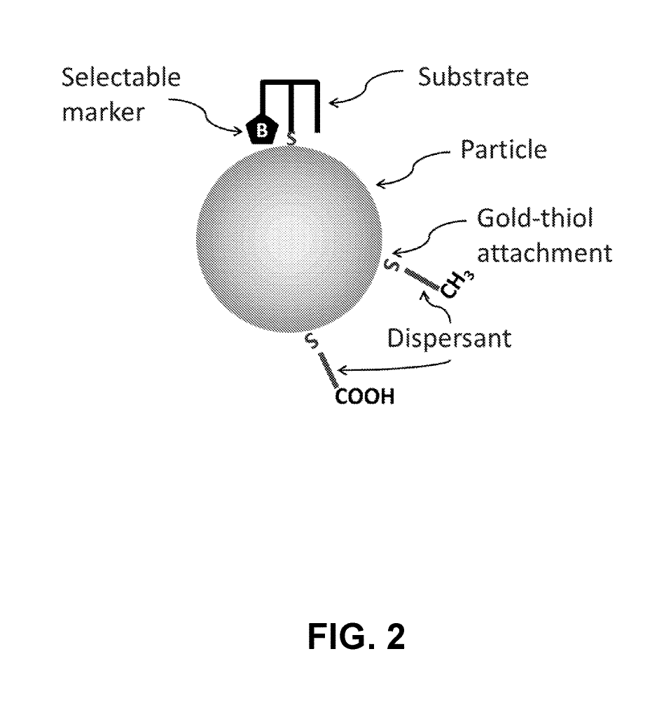

This example describes the synthesis of a lipid substrate linked to (1) a gold nanoparticle and (2) a biotin selectable marker, as shown in FIG. 2.

Ligands may be used to modify the surface of gold nanoparticles in order to serve three roles: i) to provide a means of attaching an enzyme substrate and a means of selection using a selectable marker; ii) to provide a means of dispersing the nanoparticles such that they do not aggregate; and iii) to attach DNA encoding a polypeptide. The thiolated ligands M4 (Quanta Biodesign #10799) and A12 (Quanta BioDesign #10808) provide steric and/or electrostatic repulsion between the nanoparticles. These ligands were first attached to the exterior of gold nanoparticles via ligand exchange using standard procedures (e.g. Kim, E. Y., Nucleic Acids Research, vol 34, no. 7, 2006), further described in detail below.

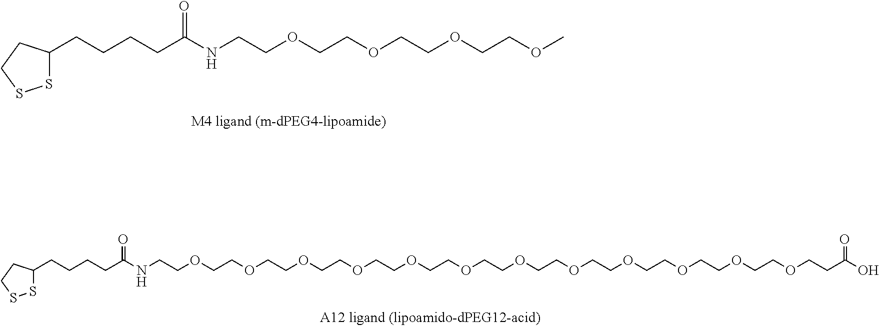 [SMILES: COCCOCCOCCOCCNC(=O)CCCCC1CCSS1 ; O=C(O)CCOCCOCCOCCOCCOCCOCCOCCOCCOCCOCCOCCOCCNC(=O)CCCCC1CCSS1]

Ligand exchanges were performed at 100 molar equivalents total (moles ligand: moles surface gold atoms), 2 mL total volume, 65% v/v THF, in 1 dram glass vials stirred at 750 rpm at 25° C. for 16 h. Briefly, a clean, dry stir bar was added to a new 20 mL glass vial (Chemglass). A12 ligand (lipoamide-dPEG12-acid) was resuspended to 10 mM in 100% THF (Acros) from 5 μmol aliquots, and was sonicated for 2 minutes in a bath sonicator (VWR 1.9 L) to speed resolubilization. M4 Ligand (m-dPEG4-lipoamide) was similarly resuspended to 10 mM in THF. LSS1b (Example 1) was resuspended to 10 mM by the addition of THF.

To the 20-mL vial, the following reactants were added under constant stirring in order: NaOH (50 μL at 2 M), THF (6 mL), M4 ligand (200 μL), A12 ligand (250 μL), LSS1b (50 μL), nanoparticles (1-mL at 5 OD-mL) and deionized water (2.95 mL). Nanoparticles were 40 nm diameter gold nanoparticles (Nanopartz A11-40). Ligand exchanges proceeded at 25° C. for 16 h.

After the exchange reaction, THF was removed by evaporation under vacuum. Exchanged nanoparticles were washed by transferring each volume to a 100 kDa spin concentrator (Millipore Amicon 15) to which 10 mL deionized water had already been added and spinning at 2,000 rcf for 1 minute in a swinging bucket centrifuge (Eppendorf 5702), pouring off the filtrate, and resuspending the retentate in 4 mL of deionized water. Washing was repeated for a total of 3 washes, and finally resuspended to a total volume of 1 mL deionized water.

Nanoparticles were quantitated by measuring optical absorption at 528 nm and the extinction coefficient provided by the manufacturer, and their concentration adjusted by dilution as needed.

The attachment of LSS1b ligands to nanoparticles was determined by attaching a DNA-probe bearing a streptavidin molecule, and quantitating that DNA-probe using quantitative PCR. Briefly, the probe DNA, which is a synthetic oligonucleotide bearing a 5′ biotin (5′ Biotin-CAAAG TATAT GCCTC TCCCC AGAGT GTTGC ACCTG TCTCC GTAGC GTCAC CTCCC GGATG GGAGA AAGTA GACTG-3′; SEQ ID NO: 1), is bound to streptavidin (ThermoFisher Pierce #21122P) at equimolar amounts at room temperature for 30 minutes in 10 mM Tris-HCl and 50 mM NaCl. The bound streptavidin-DNA-probe is then added in >10× molar excess to a small fraction of the exchanged nanoparticles in the same buffer, and allowed to bind for 30 minutes. Nanoparticles are then washed five times across a 100 kDa spin concentrator, as above, removing unbound streptavidin-probe-DNA. After washing, the number of probe-DNA's per volume is determined by quantitative PCR (qPCR) using SsoAdvanced™ Universal SYBR® Green Supermix (Bio-Rad), probe-specific primers (5′-CAAAG TATAT GCCTC TCCCC AG-3′; SEQ ID NO: 2, and 5′-CAGTC TACTT TCTCC CATCC G-3′; SEQ ID NO: 3) and LightCycler® 480 Instrument II (Roche Life Science) according to manufacturer protocols. Since all biotins (present on each LSS1b) will have a bound streptavidin-probe-DNA, this quantitation provides a measurement of the number of LSS1b ligands attached to each nanoparticle.

### Example 3: Emulsion Formation, Enzyme Digestion, and Compound Separation

Emulsions of the nanoparticles from Example 2 having 0, 5, 10, and 20% LSS1b were created and extracted in the same manner as described in U.S. Provisional Application entitled “Methods For Selecting Enzymes Having Lipase Activity” cofiled with the instant application on Apr. 7, 2015 (See, Example 6), but the exchanged nanoparticles (10⁹/emulsion) were in a solution of 50 mM HEPES, 2 mg/mL bovine serum albumin (Sigma), and Thermomyces lanuginosus lipase (TLL) (SEQ ID NO: 4) to either 0 or 10 μM final concentration.

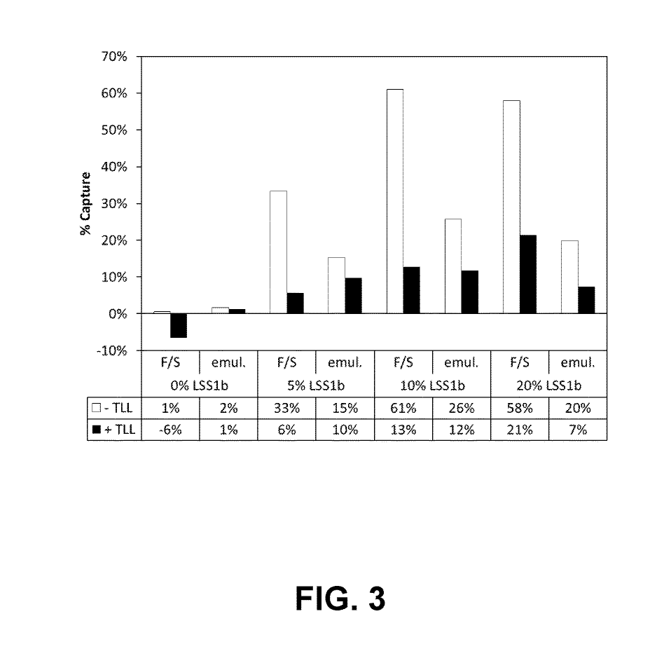

The emulsions were incubated at 30° C. for 15 minutes to allow time for lipase-mediated digestion of LSS1b, and then extracted. Identical free-solution reactions were created that omitted the emulsion formation and extraction steps. Nanoparticles were then subjected to capture using streptavidin-coated magnetic beads (Life Technologies, MyOne C1 streptavidin). Magnetic beads were removed, and the remaining nanoparticles were quantitated by measurement of optical absorbance at 528 nm. As shown in FIG. 3, digested nanoparticles (+TLL) exhibit a lower capture rate due to triglyceride hydrolysis and biotin liberation.

### Example 4: Linking DNA to Solid Phase

This example describes the bioconjugation of a polynucleotide to a gold nanoparticle using procedures as described, e.g., by Nathaniel L. Rosi et al., Science vol 312, no. 1027, 2006; Sung Yong Park, et al., Nature vol 451, no. 31 2008.

Ligand exchange reactions were performed in THF as follows: A clean, dry stir bar was added to a new 4 mL (1 dram) glass vial (Chemglass). Methyl-PEG2000-thiol ligand (#PJK-602, Creative PEGWorks; m-PEG2000-SH) was resuspended from 10 μmol aliquots (stored at −20° C.) in 100% MeOH (Acros) by adding 612 μL to make a 2000× solution (16.7 mM). Synthetic oligonucleotides, one bearing a 5′ dithiol for specific conjugation to the nanoparticles (5′ Dithiol-CAAAG TATAT GCCTC TCCCC AGAGT GTTGC ACCTG TCTCC GTAGC GTCAC CTCCC GGATG GGAGA AAGTA GACTG-3′; SEQ ID NO: 5; SR1) and one unmodified oligonucleotide to probe the possibility of non-specific conjugation to the nanoparticles (5′-GCTCC CAAAA GACCT ATCCT GACCG CAAAA TCATA ACGCC CCGAC AGCGG TGTCT TCTCC TTCCC AGTTC TTACG-3′; SEQ ID NO: 6; SR2) were used from a 100× frozen stock (0.8 mM) stored at −20° C. Ligand exchange reactions were setup by adding the amounts of THF, SR1, SR2, and NaOH indicated in Table 1 below. Vials were placed in a Pie Block (IKA) on a stir plate (IKA RET basic) and stirred at 750 rpm. Ligand exchanges were initiated by adding 1000 μL of 50 nm gold nanoparticles (Accurate A11-50, Nanopartz) to each vial, and incubated at 25° C. or 50° C. for 16 h. The indicated amount (Table 1) of m-PEG2000-SH was present either at the time of nanoparticle addition (0 m), or added 5 m after nanoparticle addition.

TABLE 1

| Reaction | A | B | C | D | E | F | G | H | I | J |
|---|---|---|---|---|---|---|---|---|---|---|
| Gold nanoparticles (μL) | 1000 | 1000 | 1000 | 1000 | 1000 | 1000 | 1000 | 1000 | 1000 | 1000 |
| 2M NaOH (μL) | 10 | 10 | 10 | 10 | 10 | 10 | 10 | 10 | 10 | 10 |
| 100x SR2 DNA (μL) | 3 | 3 | 3 | 3 | 3 | 3 | 3 | 3 | 3 | 3 |
| 100x SR1 DNA (μL) | 0 | 3 | 10 | 3 | 10 | 0 | 3 | 10 | 3 | 10 |
| 2000x m-PEG2000-SH (μL) | 50 | 50 | 49 | 50 | 49 | 50 | 50 | 49 | 50 | 49 |
| m-PEG2000-SH lag time (min) | 0 | 0 | 0 | 5 | 5 | 0 | 0 | 0 | 5 | 5 |
| 100% THF (μL) | 937 | 934 | 928 | 934 | 928 | 937 | 934 | 928 | 934 | 928 |
| Reaction temperature (° C.) | 25 | 25 | 25 | 25 | 25 | 50 | 50 | 50 | 50 | 50 |

The THF-based exchanges were transferred to 2×2 mL glass autosampler vials (Thermo) and concentrated under vacuum without heat on the high BP solvent method (Genevac EZ2 Elite, 20 m until final stage, 5 m final stage, 100 mbar reduce odor). After evaporation of the majority of the THF, 0.5 mL deionized water was added to each vial, which were then sonicated for 5 s. Samples were washed twice on 100 kDa spin concentrators (Millipore Amicon 4, UFC810024) at 2,000 rcf for 1 m in a swinging bucket centrifuge (Eppendorf 5702), by first adding 2 mL deionized water to each concentrator and then the nanoparticles. The two washes were performed by adding 4 mL deionized water and resuspending by pipetting after each spin concentration. Nanoparticles were then resuspended to a total volume of 1 mL deionized water.

For post-exchange testing, 200 μL of nanoparticles were transferred to a disposable cuvette (Brand Ultra-Micro, 759220), capped, and the absorbance spectra were measured (Nanodrop 2000c). Nanoparticle stability was assayed in 3 conditions: deionized water, 50 mM NaCl, and 50 mM NaCl+0.1M DTT. Briefly, 42.5 μL/well of either water, 0.2 M NaCl, or 0.2 M NaCl+0.4M DTT were added to a Greiner half area plate. Next, 127.5 μL of each NP sample was added and mixed by pipetting. Samples were incubated for 30 minutes at RT before measuring the absorbance at 530 and 615 nm. All fluid transfers for the Stability Assay were performed with a Biomek NXP. Each sample was then sized by DLS (Malvern Zetasizer Nano ZS) in backscatter mode using standard gold parameters (n=0.200, A=0.1). Samples were then diluted by addition of 600 μL of 50 mM HEPES, pH 7.6, loaded into a folded capillary cell (Malvern DTS1070) for zeta-potential measurement.

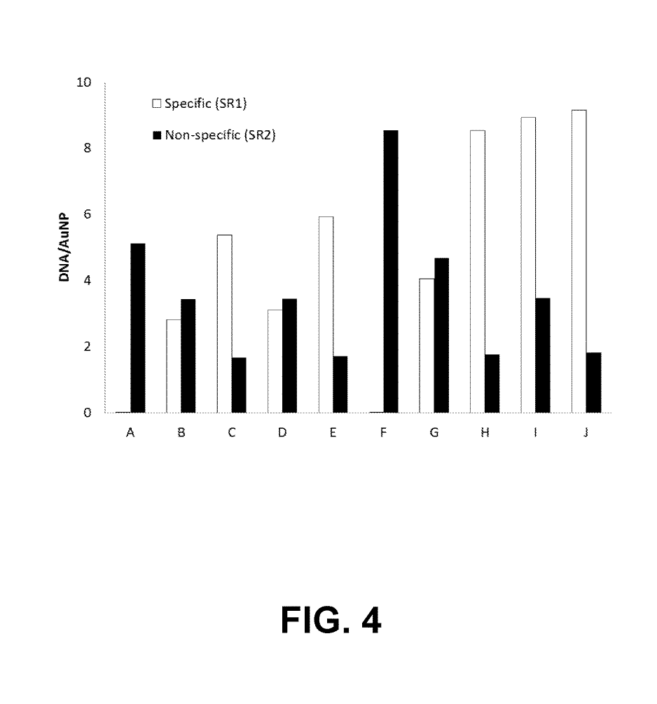

To measure specific bioconjugation of the dithiol-modified oligonucleotide (SR1) compared to non-specific binding of the unmodified oligonucleotide (SR2), washed nanoparticles were sampled for DNA quantitation. 10 μL of nanoparticles (from 1 mL final volume) were diluted 1:1000 into deionized water. 5 μL of this diluted sample were quantitated by qPCR using SsoAdvanced™ Universal SYBR® Green Supermix (Bio-Rad), probe-specific primers 5′-CAAAG TATAT GCCTC TCCCC AG-3′ (SEQ ID NO: 7) and 5′-CAGTC TACTT TCTCC CATCC G-3′ (SEQ ID NO: 8) and LightCycler® 480 Instrument II (Roche Life Science) according to manufacturer protocols. Results are presented in FIG. 4.

Although the foregoing has been described in some detail by way of illustration and example for the purposes of clarity of understanding, it is apparent to those skilled in the art that any equivalent aspect or modification may be practiced. Therefore, the description and examples should not be construed as limiting the scope of the invention.

1. A method of selecting for a polypeptide having enzyme activity, the method comprising:
(i) suspending a plurality of synthetic compounds in an aqueous phase, wherein the synthetic compounds individually comprise:
(a) a polynucleotide encoding for a polypeptide;
(b) a solid phase linked to said polynucleotide;
(c) an enzyme substrate linked to said solid phase; and
(d) a selectable marker linked to said substrate;
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with enzymatic activity toward the substrate in an aqueous droplet releases the selectable marker(s) in that droplet; and
(iv) separating the synthetic compounds to recover synthetic compounds comprising the selectable marker and/or synthetic compounds wherein the selectable marker has been released.

2. The method of claim 1, wherein no more than 20% of the aqueous droplets of the water-in-oil emulsion comprise more than one synthetic compound.

3. The method of claim 1, wherein the emulsion comprises at least about 10⁶ aqueous droplets/mL of emulsion.

4. The method of claim 1, wherein the selectable marker is an affinity tag.

5. The method of claim 1, wherein the synthetic compounds comprising the selectable marker of step (iv) are separated with streptavidin.

6. The method of claim 1, wherein the solid phase is a microbead or particle.

7. The method of claim 6, wherein the solid phase is a hydrophobic microbead.

8. The method of claim 1, wherein the solid phase is a gold nanoparticle.

9. The method of claim 1, further comprising amplifying one or more polynucleotides of the synthetic compounds wherein the selectable marker has been released of step (iv).

10. The method of claim 1, further comprising cloning one or more polynucleotides of the separated synthetic compounds from step (iv) into an expression vector.

11. The method of claim 10 further comprising transforming said expression vector into a recombinant host cell.

12. A synthetic compound comprising:
(a) a polynucleotide encoding for a polypeptide;
(b) a solid phase linked to said polynucleotide;
(c) an enzyme substrate linked to said solid phase; and
(d) a selectable marker linked to said substrate.

13. The synthetic compound of claim 12, wherein the selectable marker is an affinity tag.

14. The synthetic compound of claim 12, wherein the solid phase is a microbead or particle.

15. The synthetic compound of claim 14, wherein the solid phase is a hydrophobic microbead.

16. The synthetic compound of claim 12, wherein the solid phase is a gold nanoparticle.

17. A method of making the synthetic compound of claim 12, comprising:
(i) linking the solid phase to the polynucleotide encoding for a polypeptide;
(ii) linking the enzyme substrate to the solid phase;
(iii) linking the selectable marker to the substrate; and
(iv) recovering the synthetic compound.

18. A polynucleotide library comprising a plurality of different synthetic compounds according to claim 12.

19. A water-in-oil emulsion comprising the polynucleotide library of claim 18, wherein the synthetic compounds are compartmentalized in aqueous droplets of the emulsion.

20. A method of making the emulsion of claim 19, comprising:
(i) suspending the plurality of synthetic compounds in the aqueous phase; and
(ii) mixing the suspension of (i) with an oil.
